(19) United States
(12) Patent Application Publication
(10) Pub. No.: US 2016/0193269 A1
(43) Pub. Date: Jul. 7, 2016

(54) ANTI-DIABETIC COMPOSITION

(71) Applicant: CONOPCO, INC. D/B/A UNILEVER`, Englewood Cliffs, NJ (US)

(72) Inventors: Gautam Banerjee, Bangalore, Karnataka (IN); Nandini BALARAM SINGH, Bangalore (IN)

(73) Assignee: Conopco, Inc., d/b/a UNILEVER, Englewood Cliffs, NJ (US)

(21) Appl. No.: 14/915,724

(22) PCT Filed: Aug. 5, 2014
(86) PCT No.: PCT/EP2014/066784

§ 371 Date: Mar. 1, 2016

(30) Foreign Application Priority Data
Sep. 20, 2013 (EP) 13185257.6

(51) Int. Cl. A61K 36/28; A61K 31/4985

(52) U.S. Cl. CPC A61K 36/28; A61K 31/4985

(57) ABSTRACT

The present invention provides anti-diabetic composition comprising Inula racemosa extract and sitagliptin and a process for preparing the composition. The composition lowers blood sugar level and at the same time alleviates the negative allelo pathic effects resulting from the medication.

### FIELD OF THE INVENTION

The present invention relates to an anti-diabetic composition. More particularly the present invention relates to anti-diabetic composition comprising an Inula racemosa extract and sitagliptin and a process for preparing the composition thereof.

### BACKGROUND OF THE INVENTION

Any discussion of the prior art throughout the specification should in no way be considered as an admission that such prior art is widely known or forms part of the common general knowledge in the field.

Diabetes is one of the major and commonly occurring health problems in today's world. When diabetes is inherited from the parents it is referred to as type-1 diabetes and when it is acquired because of unhealthy life style and metabolic disorder it is referred to as type-2 diabetes. Type-2 diabetes is a condition in which blood sugar is too high because the body does not produce or use insulin normally. Whether it is type 1 or type 2, in the long term diabetes can prove to be a life threatening disease in absence of any early action to manage it.

Diabetes increases the level of dipeptidyl peptidase-4 (DPPIV) activity thereby decreasing the amount of functional glucagon-like peptide-1 (GLP-1) and glucose-dependent insulinotropic polypeptide (GIP) hormones in the circulation. One of the major roles of GLP-1 is to maintain the blood glucose level. The reduced level of GLP-1 because of increased DPPIV activity induces the imbalance and thereby increases the blood sugar level. DPPIV activity reduces levels of GLP-1 and increases blood sugar level (Hoist et.al. 1998; Inhibition of the activity of dipeptidyl-peptidase IV as a treatment for type 2 diabetes; Diabetes, Vol. 47, 1663-1670 AND Karagiannis et.al. 2012; Dipeptidyl peptidase-4 inhibitors for treatment of type 2 diabetes mellitus in the clinical setting: systematic review and meta-analysis. British Medical Journal 344).

Pharmaceutical companies are very active in this field to develop new medicines for managing diabetes. There are several medicines available in the market for treating diabetes. Pharmaceutical companies have come up with many DPPIV activity inhibitors viz. gliptins (Sitagliptin, Vildagliptin etc.) as potential solutions. However, one of the problems with diabetes is that people generally do not consider diabetes as a serious disease as there are no immediate visual effects of this disease. Therefore most of the people do not care to take any action for controlling it. Taking of medications involving alelochemicals with negative allelopathic effects is also a major concern among people, and since diabetes as such is not considered a serious disease, the medication to keep the body sugar levels under control is often neglected.

If untreated, high blood glucose levels can result in serious complications, including kidney damage (nephropathy), eye damage (retinopathy), nerve damage to the feet and other parts of the body (neuropathy), heart disease (for example, angina or heart attacks), strokes and circulation problems in the legs, sexual difficulties, including erectile dysfunction, foot ulcers or infections resulting from circulation problems and nerve damage etc. Having uncontrolled diabetes puts a person to an increased risk for developing a number of medical conditions which, if left untreated, may lead to death.

WO 2012094636 (Elcelyx Therapeutics) discloses methods for treating conditions associated with a chemosensory receptor, including diabetes, obesity, and other metabolic diseases, disorders or conditions by administrating a composition comprising a chemosensory receptor ligand, such as a bitter receptor ligand. The application also discloses chemosensory receptor ligand compositions, including bitter receptor ligand compositions, and methods for the preparation thereof and compositions comprising metformin and salts thereof and methods of use.

The paper ‘Evaluation of anti-diabetic effect of methanolic extract of Inula racemosa root in rats’ (Pharmacologyonline 3: 118-129 (2009) Mar. 8, 2009), evaluates the anti-diabetic effect of methanolic extract of Inula racemosa root in rats. On the basis of the findings of the paper, it concludes that it can be assumed that the methanolic extract of roots of Inula racemosa possess significant hypoglycaemic and antioxidant property in alloxan induced hyperglycaemia model in rats.

Generally oral anti-diabetic agents are known to have side effects and some of them are DPPIV activity inhibitors such as sitagliptin. Therefore, there is an increasing need to provide a solution which addresses the problem of lowering body blood sugar levels and at the same time alleviating the negative allelopathic effects resulting from the medication. This has been made possible by reducing the dosage of the drug and combining it with specific natural plant extracts while providing similar anti-diabetic effect.

Accordingly, one object of the present invention is to provide an effective anti-diabetic composition whereby the effective dosage of the drug sitagliptin is significantly reduced.

Another object of the present invention is to provide a process for making the anti-diabetic composition comprising sitagliptin at lower dosage levels.

### SUMMARY OF THE INVENTION

The present invention relates to a composition for anti-diabetic benefit and a process for producing the composition.

According to one aspect of the present invention there is provided an anti-diabetic composition comprising 0.5% to 2% by weight of the Inula racemosa extract and 0.000005% to 0.005% by weight of sitagliptin.

According to another aspect of the present invention there is provided a process of producing an anti-diabetic composition comprising the step of blending Inula racemosa extract and sitagliptin.

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

Except in the operating and comparative examples, or where otherwise explicitly indicated, all numbers in this description indicating amounts of material ought to be understood as modified by the word “about”.

It should be noted that in specifying any range of concentration or amount, any particular upper concentration can be associated with any particular lower concentration or amount.

For a more complete understanding of the above and other features and advantages of the invention, reference should be made to the following detailed description of preferred embodiments.

### DETAILED DESCRIPTION OF THE INVENTION

The present invention can be understood more readily by reference to the following detailed description of the invention and the examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,” “an” and “the” include plural referents unless the context clearly dictates otherwise. Thus, for example, reference to “a metal” includes mixtures of two or more metals.

The present invention relates to an anti-diabetic composition and a process for producing the composition. According to the present invention 0.5% to 2% by weight of Inula racemosa extract and 0.000005% to 0.005% by weight of sitagliptin forms an essential part of the anti-diabetic composition. According to another aspect of the present invention there is provided a process of producing the composition comprising the step of blending Inula racemosa extract and sitagliptin.

### Sitagliptin

“Sitagliptin” herein relates to the molecular structure as set out below, and is chemically known as (R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine (Chemical formula: C₁₆H₁₅F₆N₅O , Mw=407.314 g/mol), including the edible salts thereof.

[symbol]

Sitagliptin is used along with diet and exercise and sometimes with other medications to lower blood sugar levels in patients with diabetes. Sitagliptin works by inhibiting the DPPIV enzyme activity that degrades glucagon-like peptide-1 (GLP-1) and glucose-dependent insulinotropic polypeptide (GIP) hormones, allowing both to function more effectively. Sitagliptin is in a class of medications called dipeptidyl peptidase-4 (DPPIV) activity inhibitors. The mechanism of DPPIV inhibitors is to increase incretin levels (GLP-1 and GIP, inhibit glucagon release, which in turn increases insulin secretion, decreases gastric emptying, and decreases blood glucose levels.

The concentration of sitagliptin in the composition is in the range of 0.000005% to 0.005% by weight of the composition and preferably in the range of 0.00005% to 0.01% by weight of the composition.

### Inula Racemosa:

Inula racemosa is an ornamental plant of the family Asteraceae. Inula racemosa grows in the temperate and alpine western Himalayas, and it is common in Kashmir, and is also known as “Pushkarmool”. “Inula racemosa extract” herein is to be understood as a composition obtainable by preferably extracting roots of the plant or preferably parts of the roots with a solvent and more preferably with water. Herein, “extract of Inula racemosa” is the same as “Inula racemosa extract”.

The concentration of the Inula racemosa extract in the composition ranges from 0.5% to 2% by weight, and preferably ranges from 0.005% to 0.2% by weight.

The present invention provides an anti-diabetic composition comprising Inula racemosa extract and sitagliptin. The composition lowers blood sugar level and at the same time alleviates the negative allelopathic effects resulting from the medication.

One embodiment of the present invention provides a composition comprising 0.5% to 2% by weight of Inula racemosa extract and 0.000005% to 0.005% by weight of sitagliptin.

Another embodiment of the present invention provides a composition comprising 0.005% to 0.2% by weight of Inula racemosa extract and 0.00005% to 0.01% by weight of sitagliptin.

Another embodiment of the present invention provides a composition wherein the weight ratio of said sitagliptin to said Inula racemosa extract varies in range of 1:1000 to 1:40000.

Another embodiment of the present invention provides a composition wherein the weight ratio of said sitagliptin to said Inula racemosa extract varies in range of 1:20000 to 1:40000.

Another embodiment of the present invention provides a composition wherein the weight ratio of said sitagliptin to said Inula racemosa extract is 1:40000.

One embodiment of the present invention provides a composition wherein the Inula racemosa extract is an extract of Inula racemosa root or parts thereof.

One embodiment of the present invention provides a composition wherein the Inula racemosa extract is a solvent extract of the roots and preferably an aqueous extract of the roots and more preferably a dried aqueous extract of the roots.

The composition according to the invention may preferably comprise other conventional ingredients such as excipients, flavouring agents, sweetening agents etc.

The composition according to the invention is preferably formulated as edible food compositions e.g., soups, noodles, bread, jam/ketchup, beverages, ice-cream and pharmaceutical products like capsules/tablets or syrups.

In one embodiment of the present invention is provided a process of producing a composition comprising steps of blending Inula racemosa extract and sitagliptin.

In a preferred embodiment of the present invention is provided a process of producing a composition comprising steps of blending 0.5% to 2% by weight of Inula racemosa extract and by 0.000005% to 0.005% weight of sitagliptin. It is further preferred to incorporate other conventional ingredients such as acceptable excipients, flavouring agents, sweetening agents etc to obtain said composition.

One embodiment of the present invention provides a process wherein the weight ratio of said sitagliptin to said Inula racemosa extract varies in range of 1:1000 to 1:40000, preferably in the range of 1:20000 to 1:40000 and more preferably in the ratio of 1:40000.

In one embodiment, the composition of the present invention provides anti-diabetic benefit.

In one embodiment, the composition of the present invention provides an anti-diabetic benefit for type 2 diabetes.

In one embodiment, the composition of the present invention is used for the inhibition of DPPIV activity.

In one embodiment, the composition of the present invention is used for elevating GLP-1 activity.

In one embodiment the composition of the present invention is used as a medicament.

In one embodiment the composition of the present invention is used for in treating diabetes.

In one embodiment the composition of the present invention is used for inhibiting or reducing dipeptidyl peptidase-4 (DPPIV) levels.

In one embodiment is provided a method of treating diabetes by treating a person in need thereof with the composition of the present invention.

In one embodiment is provided a method of inhibiting or reducing dipeptidyl peptidase-4 (DPPIV) levels by treating a person in need thereof with the composition of the present invention.

In one embodiment the composition of the present invention is used in the preparation of a medicament for treatment of diabetes.

In one embodiment the composition of the present invention is used in the preparation of a medicament for inhibiting or reducing dipeptidyl peptidase-4 (DPPIV) levels.

Sitagliptin is a known DPPIV activity inhibitor and is being used in pharmaceutical domain. Inula racemosa is a food material being consumed widely for cardiovascular benefit. It was observed that Inula racemosa also possessed anti-diabetic benefit. It was a surprising finding of the present invention that by combining Inula racemosa extract and sitagliptin the dose of sitagliptin could be significantly reduced without altering the efficacy level.

In one embodiment, it was an unexpected finding that the composition of the present invention displayed synergistic activity and showed inhibition of DPPIV activity at a lower concentration of Sitagliptin when used in the composition than sitagliptin alone.

In one embodiment, it was a surprising finding of the present invention that the inhibition of DPPIV activity was only detectable in aqueous Inula racemosa extract and that too at a particular range of concentration, whereas the ethanolic Inula racemosa extract showed no detectable inhibition of DPPIV activity.

In another embodiment of the present invention, the inventors were successful in reducing the normal recommended effective dosage of sitagliptin prescribed for lowering blood sugar levels to significantly lower dosages and to get similar desired anti-diabetic effect.

One embodiment of the present invention provides pharmaceutical composition comprising composition of the present invention and a pharmaceutically acceptable carrier or excipient.

In one embodiment the composition of the present invention are provided as tea based beverage which include black tea based beverages, green tea based beverage and oolong tea based beverages. The preferable format may be liquid tea drink, ready-to-drink tea, tea juice etc. both hot and/or cold brew.

The edible composition of the present invention may also be in the form of a solid or powdered food supplement.

In one embodiment of the present invention is provided a process of producing an edible composition comprising the step of mixing and/or blending Inula racemosa extract and sitagliptin with the other ingredients to obtain the edible composition.

In a preferred embodiment of the present invention is provided a process of producing an edible composition comprising mixing and/or blending 0.5% to 2% by weight of Inula racemosa extract; and 0.000005% to 0.005% by weight of sitagliptin with the other ingredients to obtain the edible composition.

In another more preferable embodiment is provided a process of producing an edible composition comprising mixing and/or blending 0.005% to 0.2% by weight of Inula racemosa extract; and 0.00005% to 0.01% by weight of sitagliptin with the other ingredients to obtain the edible composition.

The term “other ingredients” as mentioned above means the compositional ingredients needed for making a targeted edible product e.g. in case of making a soup composition (targeted edible product) the term “other ingredients” preferably are starch, salt, sugar, yeast extract, fat powder, vegetable pieces, flavouring agents, colour etc.

To make the edible composition of the present invention, the Inula racemosa extract may be prepared by extracting (boiling) the roots of Inula racemosa with preferably water at a temperature in the range of 70° C. to 100° C. for 2-6 hours followed by cooling. After that the solution is preferably filtered and concentrated. The concentration stage preferably carried out in a rotary evaporator.

Alternately, commercially available Inula racemosa aqueous extract powder may also be used.

Without wishing to be bound by theory it is stated that type-2 diabetes is associated with increasing DPPIV activity and thereby decreasing GLP-1 and GIP activity. GLP-1 generally maintains the balance by controlling blood sugar level. Increasing level of DPPIV activity suppresses the activity of GLP-1 and GIP. The present invention is preferably developed to inhibit DPPIV activity and thereby maintaining the activity of GLP-1 which in turn controls the blood sugar level.

Further details of the invention, its objects and advantages are explained here under in greater details with reference to the following non-limiting examples. It would be apparent to a person skilled in the art that many such examples are possible and the examples given under are for illustrative purpose only. These should not be construed so as to limit the scope of this invention in any manner.

### EXAMPLES

### Example 1: The Effect of Different Concentrations of Sitagliptin on the Activity of DPPIV Enzyme.

Different concentrations of sitagliptin were tested for its effect on DPPIV activity in an in vitro assay. DPPIV activity is measured by the ability of the DPPIV enzyme to cleave the chromogenic substrate Gly-Pro-p-nitro anilide. The cleaved product p-nitro aniline is measured at 405 nm in a Micro Plate reader.

In the different wells of the 96 well plate (NEST Biotechnology Co. Ltd, Cat No 701001) 10I of various concentrations of sitagliptin ranging from 0.000005% to 0.005% as per Table 1 were added. In each well, 10·I of human serum which contains DPPIV enzyme was also added. The final volume was made up to 200 μl/well using TRIS-HCl buffer of pH-8. The TRIS-HCl buffer was prepared by adding 2.42 g of TRIS (Tris hydroxyl methyl amino methane; Supplier: Sisco Research Laboratory ltd, Cat No 2044122) base, 0.372 g of EDTA (SIGMA, Cat No E6758) and 5.644 g of NaCl in 900 mL of autoclaved milli-Q water (Millipore® India) and stirred till it is dissolved. After that the pH of the solution was adjusted to 8.0 using HCl acid and then the volume of the solution was made up to 1000 mL with autoclaved milli-Q water.

The serum and the sitaglipin solution at various concentrations were mixed for 1 min using microplate reader (BIO-RAD LAB INDIA, Model No. 680) and incubated in an incubator at 37° C. (Thermo Scientific, Model 3111) for 10 minutes. After that the enzymatic reaction was initiated by adding 10 μL/well of the 19 mM of substrate Glycine-Proline para nitroanilide (Gly-Pro p- NA) (this is equivalent to GLP-1). Gly-Pro-p NA is a universally accepted and commercially available substrate for the enzyme DPPIV. (Yogisha et. al., Journal of Natural Products, Vol. 3(2010):76-79).

The above reaction mixture was incubated for 1 hour in the incubator at 37° C. Then the enzymatic activity was arrested by adding 100 μL/well of citrate buffer of pH 4. The citrate buffer was prepared by adding 2.1g of citric acid monohydrate (Sisco Research laboratory ltd, Cat No 0348216) and 2.94 g of Sodium citrate tri basic dihydrate (Sisco Research laboratory ltd, Cat No 1949110) in 90 mL of autoclaved milli-Q water (Millipore® India). After that the pH of the solution was adjusted to 4.0 using HCl acid and then the volume of the solution was made up to 100 mL with autoclaved milli-Q water. After this the absorbance was measured at a wavelength of 405 nM using microplate reader (BIO-RAD LAB INDIA, Model No. 680).

Combination of Gly-Pro-p NA and serum at the same respective concentration used above was maintained as a control.

The % activities of the DPPIV for the samples were calculated using the following formula:

$$\frac{O . D of the sample}{O . D of the control} \times 100 = \% Activity$$

The % inhibitions by the samples were then calculated by subtracting the % activity from 100. The results of the experiments summarized below in Table 1.

TABLE 1

| Sitagliptin (% weight) | % Inhibition of DPPIV activity |
|---|---|
| 0.000001 | Not Detected |
| 0.000005 | 12.57 |
| 0.00001 | 32.24 |
| 0.00002 | 47.15 |
| 0.00005 | 64.14 |
| 0.0005 | 80.50 |
| 0.005 | 86.32 |

Data presented in Table 1 show that the inhibition of DPPIV enzyme activity increased in a dose dependent manner.

### Example 2: The Effect of Extracts of Inula Racemosa in Different Solvents on the Activity of DPPIV Enzyme.

Inula racemosa extract was prepared by using the following procedure:

The Inula racemosa (pushkarmool) plant was bought from the local (Bangalore, India) market. This was available as a stem size of 3 cm to 6 cm which was a combination of roots and stems of pushkarmool. The dried Inula racemosa powder using only the roots was prepared by a pulverizing the material using a cutting mill, Retsch SM 100 to which was attached a 200 μm size sieve.

Inula racemosa extract was prepared from dried Inula racemosa powder. 100 g of dry Inula racemosa root powder was soaked in of water or ethanol for about 14 hours and then boiled at 80° C. for 4 hours for obtaining aqueous or ethanolic extracts respectively. It was then cooled down to about 35° C. followed by filtering the solution to get a clear solution. The solution was then concentrated to dryness (moisture content of about 3%) using rotary evaporator (Heidolph Laborota 4002). This extract was used for the experiments as described below.

Various concentrations of aqueous and ethanolic extracts of Inula racemosa as shown in Table 2 were tested for its effect on DPPIV activity in an in vitro assay as disclosed in Example 1.

TABLE 2

| | % Inhibition of DPPIV activity | |
|---|---|---|
| Inula racemosa % weight | Aqueous Extract | Ethanolic extract |
| 0.00004 | ND (Not detected) | ND |
| 0.0002 | ND | ND |
| 0.0004 | ND | ND |
| 0.002 | ND | ND |
| 0.004 | ND | ND |
| 0.005 | 4.8 | ND |
| 0.02 | 5.0 | ND |
| 0.05 | 5.2 | ND |
| 0.1 | 4.12 | ND |
| 0.2 | 13.24 | ND |

The results presented in Table 2 show that the aqueous Inula racemosa extract at lower concentrations did not show detectable DPPIV inhibitory activity while starting from 0.005% detectable level of inhibition was observed. The highest inhibition was observed at 0.2% aqueous Inula racemosa extract. However, the ethanolic extract did not show any detectable DPPIV inhibitory activity at all concentrations of Inula racemosa extract tested.

The higher concentrations of both aqueous and ethanolic extracts of Inula racemosa (above 0.2%) were also tested for the inhibition of the activity of DPPIV, but it was found that this lead to acidification of the reaction mixture which indicates self-deactivation of the DPPIV enzyme. Therefore those concentrations are not mentioned in the table.

### Example 3: Effect of a Combination of Inula Racemosa Extract and Sitagliptin on the Activity of DPPIV Enzyme.

Aqueous Inula racemosa extract was prepared as described in Example 2.

The in vitro DPPIV enzyme assay was performed by using various concentrations as indicated in Table 3 of Inula racemosa aqueous extract alone and in combination with sitagliptin using the procedure described for Example 1. Sitagliptin alone was also evaluated for comparison. The concentration of sitagliptin was selected at 0.000005% based on the results of Example 1. The concentration of Sitagliptin chosen was the minimum concentration at which the Inhibition of DPPIV activity was detected. Different concentrations of aqueous Inula racemosa extract were selected based on the results of Example 2 starting from the minimum concentration of Inula racemosa extract at which the Inhibition of DPPIV activity was detectable. The data on % inhibition of DPPIV activity is presented in Table 3.

TABLE 3

| (% weight) | | % Inhibition of DPPIV activity |
|---|---|---|
| Sitagliptin | 0.000005 | 9.67 |
| Inula Racemosa | 0.005 | 5.86 |
| | 0.05 | 9.62 |
| | 0.2 | 16.63 |
| Inula | 0.005(IR) + 0.000005(S) | 18.37 |
| Racemosa(IR) + | 0.05(IR) + 0.000005(S) | 20.56 |
| Sitagliptin(S) | 0.2(IR) + 0.000005(S) | 31.70 |

Data presented in Table 3 show that a combination of Inula racemosa extract and sitagliptin exhibited synergistic effect in terms of the inhibition of DPPIV activity as compared to the individual effects of Inula racemosa or sitagliptin.

The present invention thus provides for a synergistic composition comprising Inula racemosa extract and sitagliptin which is an effective anti-diabetic composition where the dosage of the drug sitagliptin is significantly reduced.

1. An anti-diabetic composition comprising:
i. 0.5% to 2% by weight of Inula racemosa extract, and
ii. 0.000005% to 0.005% by weight of sitagliptin.

2. The composition as claimed in claim 1, wherein the Inula racemosa extract ranges from 0.005% to 0.2% by weight of the composition.

3. The composition as claimed in claim 1, wherein the sitagliptin ranges from 0.00005% to 0.01% by weight of the composition.

4. The composition as claimed in claim 1 wherein the weight ratio of said sitagliptin to said Inula racemosa extract varies in range of 1:1000 to 1:40000.

5. The composition as claimed in claim 1 wherein the weight ratio of said sitagliptin to said Inula racemosa extract varies in range of 1:20000 to 1:40000.

6. The composition as claimed in claim 1 wherein the Inula racemosa extract is an extract of Inula racemosa root or parts thereof.

7. The composition as claimed in claim 1 wherein the Inula racemosa extract is an aqueous extract.

8. A process of producing a composition according to claim 1 the process comprising the step of blending Inula racemosa extract and sitagliptin.

9. A composition according to claim 1 for use as a medicament.

10. A composition according to claim 1 for use in treating diabetes.

11. A composition according to claim 1 for use in treating diabetes by inhibiting or reducing dipeptidyl peptidase-4 (DPPIV) levels.

12. A method of treating diabetes by treating a person in need thereof with a composition according to claim 1.

13. A method of treating diabetes by inhibiting or reducing dipeptidyl peptidase-4 (DPPIV) levels by treating a person in need thereof with a composition according to claim 1.

14. Use of a composition according to claim 1 in the preparation of a medicament for treatment of diabetes.

15. Use of a composition according to claim 1 in the preparation of a medicament for treatment of diabetes by inhibiting or reducing dipeptidyl peptidase-4 (DPPIV) levels.
